(19) United States
(12) Patent Application Publication
(10) Pub. No.: US 2010/0076251 A1
(43) Pub. Date: Mar. 25, 2010

(54) PYRO/PIEZO SENSOR AND STIMULATOR

(75) Inventors: Peter Stasz, Mounds View, MN (US); Reinhold Henke, Plymouth, MN (US)

Correspondence Address: SCHWEGMAN, LUNDBERG & WOESSNER, P.A., P.O. BOX 2938, MINNEAPOLIS, MN, 55402, US

(73) Assignee: Dymedix Corporation, Shoreview, MN (US)

(21) Appl. No.: 12/562,669

(22) Filed: Sep. 18, 2009

Related U.S. Application Data
(60) Provisional application No. 61/098,398, filed on Sep. 19, 2008.
(60) Provisional application No. 61/098,394, filed on Sep. 19, 2008.

(51) Int. Cl. A61M 21/02

(52) U.S. Cl. 600/28; 600/27; 600/26

(57) ABSTRACT

This document discusses, among other things, an apparatus and method for detecting respiration information of a patient and for providing a stimulation to the patient. A sleep sensor transducer includes a pyro/piezoelectric film. A first and second electrode can attach to the film to transmit the detected respiration information to a closed loop neuromodulator and to receive stimulation energy from the closed loop neuromodulator to provide the stimulation.

### PRIORITY AND RELATED APPLICATIONS

This application claims the benefit under 35 U.S.C. 119(e) of U.S. Provisional Patent Application Ser. No. 61/098,394, filed on Sep. 19, 2008, and U.S. Provisional Patent Application Ser. No. 61/098,398, filed Sep. 19, 2008, the disclosures of which are incorporated by reference herein in their entirety.

### TECHNICAL FIELD

The present subject matter relates generally to the field of neurological disorders and more specifically to the area of sleep medicine and yet even more specifically to the area of sleep therapy for patients who suffer from sleep disorders. More particularly, the present invention relates to a device for sensing airflow, snoring and effort and stimulating the central nervous system of a patient suffering from one or multiple sleep disorders.

### BACKGROUND

Sleep disorders have recently become the focus of a growing number of physicians. Sleep disorders include obstructive sleep apnea, central sleep apnea, complex sleep apnea, snoring, restless leg syndrome (RLS), periodic limb movement (PLM), sudden infant death syndrome (SIDS), and related neurological and physiological events or conditions occurring during sleep.

Many hospitals and clinics have established sleep laboratories (sleep labs) to diagnose sleep disorders. In the sleep laboratories, practitioners use instrumentation to monitor and record a patient's sleep states, stages and behaviors during sleep. Practitioners rely on these recordings to diagnose patients and prescribe proper therapies. As a result of the sleep diagnosis in the sleep laboratory, the practitioner prescribes a sleep therapy device for the patient to use during periods of regular sleep at home.

A goal of addressing sleeping disorders is to help a person sleep better. Another goal is to help a person live longer. It is well known that various undesirable behaviors often occurring during sleep include snoring, apnea episodes, abnormal breathing, Bruxism (teeth clenching and grinding) and the like. It is further known that these disorders and other undesirable behaviors can not only lead to insufficient amounts of sleep and resulting fatigue, but are also linked to co-morbidities, such as obesity, high blood pressure, diabetes, cardiac diseases and stroke, all of which lead to a pre-mature death. Even SIDS is suspected to be linked to an infant's sleep disorder.

Another goal of addressing sleeping disorders is often to help a person sleep better. It is well known that several states of sleep exist and involve varying levels of consciousness. It is further well known that the beneficial effects of sleep improve when it is uninterrupted. To the extent that devices alter a patient's sleep state, or in a worst-case scenario, actually awaken a patient, the devices have gone too far. While they may have stopped the undesirable behavior, they have not helped a person sleep better.

Serious efforts are being made to reduce or eliminate these undesirable disorders and behaviors in part because of these co-morbidity concerns.

### SUMMARY

Certain embodiments of the present invention provide a sensor and stimulation device that by means of a piezoelectric to mechanical motion conversion provides stimulation of the central nervous system in general by means of tactile stimulation.

In various examples, the pyro/piezo sensor and stimulator comprise a small strip of metallized piezoelectric PVDF film, with wire terminals attached to opposing metallized surfaces. The film and the wire terminals are further sandwiched on one side with a single sided adhesive tape of approximately the same size of the piezoelectric film and on the other side with a double sided adhesive tape of approximately the same size of the piezoelectric film so it can be affixed to the patient's skin for the duration of the therapy.

In Example 1, a sleep therapy transducer for a patient includes a pyro/piezoelectric film configured to detect respiration information of the patient and configured to provide a stimulation to the patient, a first electrode attached to a first major surface of the pyro/piezoelectric film, a second electrode attached to a second major surface of the pyro/piezoelectric film, wherein the first and second electrodes are configured to transmit the detected respiration information from the pyro/piezoelectric film to a closed loop neuromodulator and to receive stimulation energy from the closed loop neuromodulator, and wherein the stimulation is provided to the patient using the received stimulation energy.

In Example 2, the sleep therapy transducer of Example 1 optionally includes a first adhesive to attach the first electrode to the first major surface of the pyro/piezoelectric film.

In Example 3, the sleep therapy transducer of any one or more of Examples 1-2 optionally include a second adhesive to attach the second electrode to the second major surface of the pyro/piezoelectric film.

In Example 4, the first and second adhesives of any one or more of Examples 1-3 optionally include a conductive adhesive.

In Example 5, the sleep therapy transducer of any one or more of Examples 1-4 optionally include a first wire coupled to the first electrode and a second wire coupled to the second electrode, the first and second wires configured to transmit detected respiration information from the pyro/piezoelectric film to a closed loop neuromodulator and to transmit stimulation energy from the closed loop neuromodulator to the pyro/piezoelectric film.

In Example 6, the sleep therapy transducer of any one or more of Examples 1-5 optionally includes a first adhesive film substantially covering the first major surface of the pyro/piezoelectric film, the first adhesive film to secure the first wire to the first major surface of the pyro/piezoelectric film, and a second adhesive film substantially covering the second major surface of the pyro/piezoelectric film, the second adhesive film to secure the second wire to the second major surface of the pyro/piezoelectric film.

In Example 7, the first adhesive film of any one or more of Examples 1-6 is optionally a double sided adhesive film configured to secure the sleep therapy transducer to the patient.

In Example 8, the double sided adhesive film of any one or more of Examples 1-7 is optionally configured to provide acoustic impedance matching between the sleep therapy transducer and the patient's skin.

In Example 9, the pyro/piezoelectric film of any one or more of Examples 1-8 optionally includes a metalized polyvinylidene fluoride (PVDF) film.

In Example 10, the sleep therapy transducer of any one or more of Examples 1-9 is optionally configured to attach behind an ear of the patient.

In Example 11, the sleep therapy transducer of any one or more of Examples 1-10 is optionally configured to attach near a lip of the patient.

In example 12, the sleep therapy transducer of any one or more of Examples 1-11 is optionally configured to attach over the patient's nose.

In Example 13, the sleep therapy transducer of any one or more of Examples 1-12 is optionally configured to attach to the patient's neck.

In Example 14, the sleep therapy transducer of any one or more of Examples 1-13 is optionally configured to emit at least one of a tactile stimulation or an acoustic stimulation.

In Example 15, a sleep therapy transducer for a patient includes a pyro/piezoelectric film configured to detect respiration information of the patient and configured to provide a stimulation to the patient, a first electrode attached to a first major surface of the pyro/piezoelectric film, a second electrode attached to a second major surface of the pyro/piezoelectric film, a first conductive adhesive to attach the first electrode to the first major surface of the pyro/piezoelectric film, a second conductive adhesive to attach the second electrode to the second major surface of the pyro/piezoelectric film, a first wire coupled to the first electrode and a second wire coupled to the second electrode, a first adhesive film substantially covering the first major surface of the pyro/piezoelectric film, the first adhesive film to secure the first wire to the first major surface of the pyro/piezoelectric film, a second adhesive film substantially covering the second major surface of the pyro/piezoelectric film, the second adhesive film to secure the second wire to the second major surface of the pyro/piezoelectric film, wherein the first and second wires are configured to transmit detected respiration information from the pyro/piezoelectric film to a closed loop neuromodulator, and to transmit stimulation energy from the closed loop neuromodulator to the pyro/piezoelectric film, and wherein the first adhesive film is a double sided adhesive film configured to secure the sleep therapy transducer to the patient.

In Example 16, a method includes detecting respiration information of a patient using a sleep therapy transducer attached to the patient, the sleep therapy transducer including a pyro/piezoelectric film, transmitting the detected respiratory information from the sleep therapy transducer to a closed loop neuromodulator using a first electrode attached to a first major surface of the pyro/piezoelectric film and a second electrode attached to a second major surface of the pyro/piezoelectric film, receiving stimulation energy from the closed loop neuromodulator using the first and second electrodes, and providing a stimulation to the patient using the received stimulation energy.

In Example 17, the transmitting the respiration information of Example 16 optionally includes transmitting the respiration information using a using a single wire pair coupled to the first and second electrode.

In Example 18, the receiving the stimulation energy of any one or more of Examples 16-17 optionally includes receiving the stimulation energy using the single wire pair coupled to the first and second electrode.

In Example 19, the providing the stimulation of any one or more of Examples 16-18 optionally includes providing the stimulation behind an ear of the patient.

In Example 20, the providing the stimulation of any one or more of Examples 16-19 optionally includes providing the stimulation near a lip of the patient.

In Example 21, the providing the stimulation of any one or more of Examples 16-20 optionally includes providing the stimulation to the patient's nose.

In Example 22, the providing the stimulation of any one or more of Examples 16-21 optionally includes providing the stimulation to the patient's neck.

In Example 23, the providing the stimulation of any one or more of Examples 16-22 optionally includes providing at least one of tactile stimulation or acoustic stimulation.

While the present disclosure is directed toward treatment of sleep disorders, further areas of applicability will become apparent from the description provided herein. It should be understood that the description and specific examples are intended for purposes of illustration only and are not intended to limit the scope of the invention.

### BRIEF DESCRIPTION OF THE DRAWINGS

The forgoing features, objects and advantages of the invention will become apparent to those skilled in the art from the following detailed description, especially when considered in conjunction with the accompanying drawings in which like the numerals in the several views refer to the corresponding parts:

FIG. 1 is a configuration diagram of a pyro/piezo sensor and stimulator in place on a patient according to one embodiment of the present invention;

FIG. 2 is a partial peel away view of the mechanical outline of a pyro/piezo sensor and stimulator according to one embodiment of the present invention;

FIG. 3 is an exploded, greatly enlarged edge view of the mechanical outline of a pyro/piezo sensor and stimulator according to one embodiment of the present invention;

### DETAILED DESCRIPTION

The following detailed description relates to a pyro/piezo sensor and stimulator to be used for treating patients suffering from sleep disorders. The pyro/piezo sensor and stimulator is more particularly directed at stimulating a patient to interrupt and terminate an undesired sleep behavior or condition, such as snoring, sleep apnea, sudden infant death syndrome (SIDS) and others. The pyro/piezo sensor and stimulator may be used in conjunction with a sensor affixed to a patient and a controller coupled to receive a sensor output. The sensor transmits respiratory information to a controller that analyzes the information and capable of energizing the pyro/piezo sensor and stimulator depending on the information received from the controller.

The assignee of the present invention, Dymedix Corporation of Shoreview, Minn. has earlier filed a U.S. Provisional Patent Application titled “Agitator to Stimulate the Central Nervous System”, Ser. No. 61/049,802, filed May 2, 2008, the contents of which are hereby incorporated by reference herein.

Various stimulation systems are available for altering undesirable behavior during sleeping. For example, several United States patents are directed toward treatment of snoring including U.S. Pat. No. 4,644,330 to Dowling, U.S. Pat. No. 4,788,533 to Mequignon, and U.S. Pat. No. 5,477,867 to Balkanyi. These patents rely on an “aversive audio stimulus,” a “sound stimulus,” and “acoustic signals”, respectively.

As another example, in U.S. Pat. No. 3,802,417 to Lang, an alarm relay is triggered “if the desired standard respiratory frequency is not attained. When triggered, the alarm relay switches on a respiration stimulator that consists of a rhythmically inflatable belt or cuff.”

As an additional example, U.S. Pat. No. 4,715,367 to Crossley discloses a device “to diagnose, treat and monitor the treatment for snoring, bruxism, or sleep apnea. Treatment consists of regulatable aversive shock.”

In still another example, U.S. Pat. No. 6,093,158 to Morris discloses “monitoring an undesired behavioral disorder, such as bruxism, jaw clenching, or snoring.” The patent describes, inter alia, “an alert system selected from the group consisting of light controllers, temperature regulators, odor generators, high frequency generators, tactile pressure generators, vibratory generators, and electric current generators.”

U.S. Pat. No. 6,935,335 to Lehrman et al. claims “creating a stimulus to said person's neck muscles to cause said person to move said person's neck muscles to move said person's head backwards to terminate said physiological process before cessation of breathing occurs.” The stimulus is further claimed as “a vibrator and a sound generator placed adjacent to said person's neck muscles.” Additional examples can be found in U.S. Patent Publication No. US2003/0195588 to Fischell et al. and US2008/0009915 to Moses et al.

In light of the fine line between interrupting an undesirable behavior and altering a sleep state, there are several problems associated with the above devices. First, in the case of the audio, light, or odor devices, it is well known that a vast range of patient sensitivities exist for each of these stimuli. Thus, these devices have difficulty simultaneously altering an undesirable behavior and avoiding alteration of a sleep state because they are often under or over effective at stimulating a patient. Second, the inflatable belt or cuff and the temperature regulator both involve a certain ramp up and shut down time. This can make it difficult to precisely dose a patient when trying to stimulate the patient at the instant an undesirable sleep behavior starts and to stop stimulation at the instant the behavior stops. Third, regarding the electric current and aversive shock, many people simply are not comfortable with the nature of this treatment. Fourth, vibration can work fairly universally, however, due to the inertial forces involved, the devices tend to have the same shut down problems mentioned above. That is, once the undesirable behavior stops and the device is switched off, it takes time for the vibration to stop and thus stop stimulating the patient. This can lead to over stimulation and an alteration in the patient's sleep state.

The inventors have recognized needs, and have developed apparatus and methods, for better sleep therapy devices including, but not limited to, a sensor and stimulator capable of sensing respiratory events and stimulating the central nervous system in a single device, a sensor and stimulator capable of sensing respiratory events and stimulating the central nervous system comprising a single wire pair for sensing and stimulation, a sensor and stimulator capable to sufficiently interrupt an undesirable sleep behavior by a stimulus universally sensed by most patients where the stimulation device avoids significantly changing sleep states and certainly avoids waking a patient, a sensor and stimulator controllable so as to provide precise doses of stimulation to a sleep disorder patient and an apparatus that is comfortable to wear so as to further its ability to avoid alteration of sleep states.

Various examples described below include a pyro/piezo sensor and stimulator device including a PVDF film with piezoelectric and pyroelectric properties, sandwiched between a single sided adhesive film layer and a double-sided adhesive film layer, and being in contact with a sleep patient. The PVDF film is connected to a source of alternating current of a selected frequency and amplitude such that when activated, the film rapidly expands and contracts creating a mechanical vibration. This creates a tactile event, which is detected by the central nervous system of the sleep patient. The pyro/piezo sensor and stimulator is such as to interrupt undesired sleeping behavior, but the patient's sleep states are not altered nor is the patient awakened. The film is further disclosed as a piezoelectric film or a polyvinylidene film with metallized opposing surfaces.

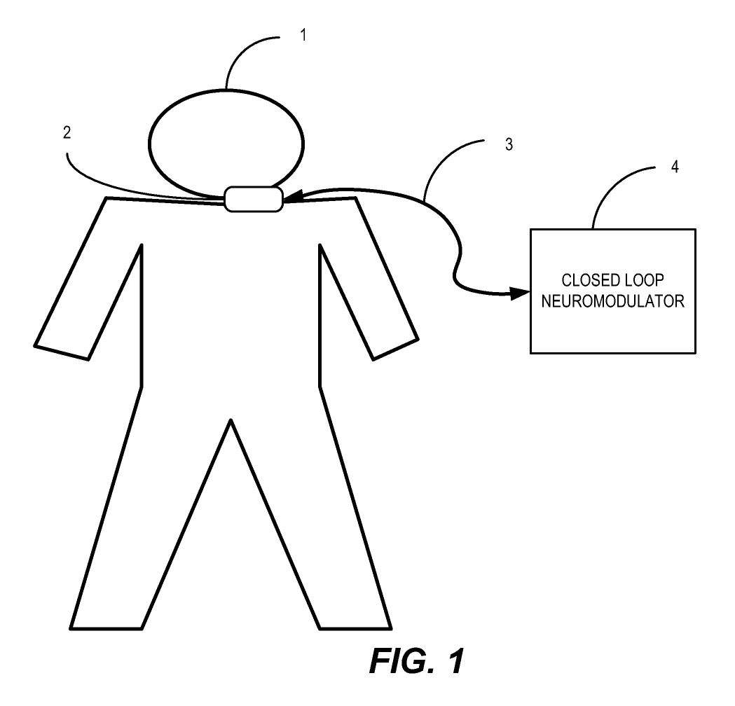

The following detailed description also includes discussion of sensors affixed to patients, controllers, and transducers. Additionally, elements of a pyro/piezo sensor and stimulator are discussed including a piezo-film, wire terminations, a single sided adhesive tape, and a double-sided adhesive tape encasing the film. The present invention can be readily understood from FIGS. 1 through 3.

Referring to FIG. 1, there is indicated generally by numeral 1 a typical sleep therapy patient who has been outfitted with a pyro/piezo sensor and stimulator 2 to measure respiratory activity and to stimulate the patient by applying sleep therapy dosages. A pair of pyro/piezo sensor and stimulator output wire leads 3 connect the pyro/piezo sensor and stimulator to the sense and stimulation input/output terminals of a closed loop neuromodulator 4.

In various examples, the pyro/piezo sensor is configured to attach to the patient for sensing information, such as respiration information, and for stimulating the patient's central nervous system to interrupt a sleep disorder event without altering a sleep state of the patient. The pyro/piezoelectric sensor and stimulator may be placed in a number of locations on a patient including, but not limited to, behind or near the patient's ear, over the patient's nose, on a patient's neck across the throat or above or below a patient's lips.

Having described the overall configuration of pyro/piezo sensor and stimulator with the aid of FIG. 1, a more detailed explanation of a specific implementation of the pyro/piezo sensor and stimulator will now be presented.

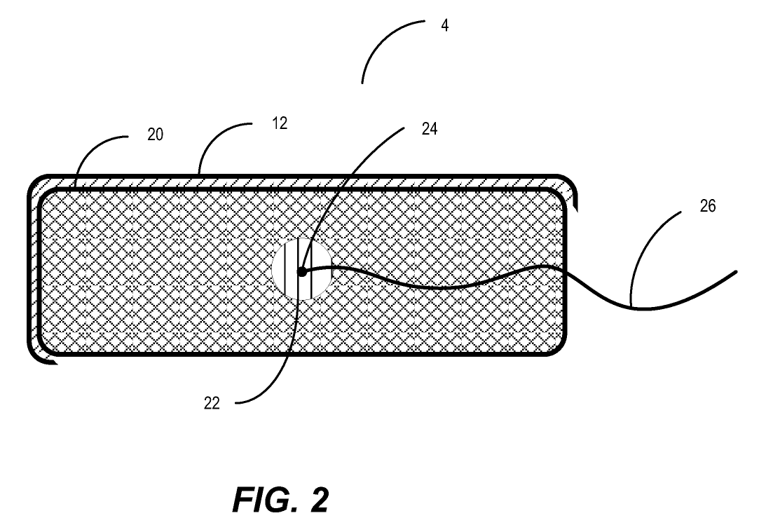

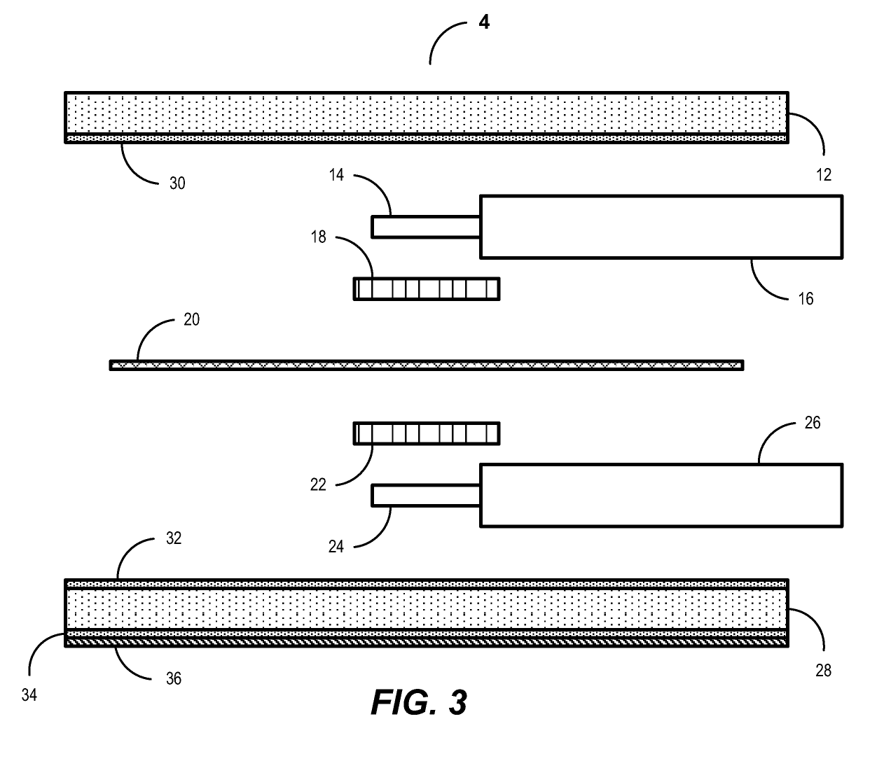

Referring to FIGS. 2 and 3, there is shown a partial peel away view and an enlarged exploded edge view of a pyro/piezo sensor and stimulator. The pyro/piezo sensor and stimulator comprises as its active element one PVDF film 20. The PVDF film 20 includes metallization layers on opposed major surfaces thereof represented by the cross-hatching thereon. The metallization layers serve to apply a drive signal across the film layer 20 to generate motion or vibration in response to the control signal delivered by a sleep therapy device. In an example, the sleep therapy device can include a closed loop neuromodulator, such as that described in U.S. patent application Ser. No. 12/583,581 filed Aug. 21, 2009, the entire disclosure of which is incorporated herein by reference.

To insure intimate contact between the conductive copper of the wires 14 and 24 and the metallized surfaces of the pressure sensing PVDF film transducer 20, a conductive adhesive, such as that sold under the trademark ARclad® by Adhesives Research, Inc., is used. This material comprises an adhesive that is laced with conductive carbon particles that serves as a bonding agent between the exposed copper 14 and 24 of wires 16 and 26 with the metallized layers adhered to the PVDF film transducer. The ARclad® adhesive is represented in FIG. 2 by numerals 18 and 22.

A first single-sided adhesive tape 12 with adhesive layer 30 cut to conform to the shape of the vibration generating PVDF film transducer 20 and adheres to a first of the opposed surfaces of the film layer 20, helping to secure the copper portion 14 of the wire lead 26.

A second double-sided adhesive tape 28 with adhesive layer 32 and 34 has the outer adhesive layer 34 protected by an outer adhesive layer protection film 36 cut to conform to the shape of the vibration generating PVDF film transducer 20. The layer 32 is adhered to the opposed surfaces of the film layer 20, helping to secure the copper portion 24 of the wire lead 26. The second double-sided adhesive tape layer 34 also acts as an acoustic impedance matching layer between the pyro/piezo sensor and stimulator and the patient's skin after the protective film 36 has been removed and the drive applied to an area of exposed skin.

The film shown in FIGS. 1-3 may be a piezoelectric film. More particularly, the film may preferably be a polyvinylidene fluoride (PVDF) film. As mentioned, the PVDF film may be a metallized PVDF film of either series or parallel bimorphic structure. Those skilled in the art will understand and appreciate the piezoelectric to mechanical motion conversion properties of PVDF. However, other films known in the art, which have mechanical motion conversion properties, are within the scope of the invention.

The resulting mechanical vibration of the film is transferred to the patient's skin where the pyro/piezo sensor and stimulator both creates a tactile and audible sensation. This mechanical vibration is passed subsequently to the patient's tissue that the pyro/piezo sensor and stimulator is in contact with. This transfer of mechanical vibration via the patient's skin to the patient's tissue creates a tactile event, which is detected by the central nervous system.

In an example, the stimulation of the pyro/piezo sensor and stimulator is sufficient to cause a sleeping patient to interrupt an undesirable sleeping behavior, but is not sufficient to alter the sleep state of the patient. Thus, depending on the condition being treated, the patient may stop snoring, or start breathing without alteration of the patient's sleep state.

The nature of the thin film and lightweight housing allows the device to maintain a low momentum even at high velocity. Thus, the ramp up and shut down period required for this device is almost instantaneous. When the AC excitation voltage is transmitted, the device immediately begins its mechanical vibration and when the AC excitation voltage stops, the device immediately stops. This is because the momentum of the device, while moving very quickly, is small, because of its low mass. This is in contrast to well known vibratory devices in cell phones and pagers, where the mass of the devices is quite large relative to the pyro/piezo sensor and stimulator disclosed herein.

The present invention is advantageous because it is directed toward transfer of mechanical energy to create a tactile sensation, which makes the device universally effective for most sleep patients. This is in contrast to the sound, light, and odor type devices of the prior art.

An additional advantage of the present invention is the precision with which stimulating doses can be given to a sleep patient. Due to the light weight nature of the device and its electrical activation, the device can be started and stopped extremely quickly, thus stimulating the central nervous system in very specific and defined doses. This contributes to its ability to interrupt an undesired behavior and yet avoid altering the patients' sleep state.

Another advantage of the present invention relating to its light weight is the ability to cause considerable mechanical vibration with very little electrical power simply by stimulating the device with an alternating current. This advantage makes it possible for the controller device to be supplied with battery power and to maintain battery life for an extended period of time.

Another advantage of the present invention is the ability to sense and stimulate the central nervous system in a single device.

Another advantage of the present invention is the capability of sensing respiratory events and stimulating the central nervous system comprising a single wire pair for sensing and stimulation.

Another advantage of the present invention is the ability to sufficiently to interrupt an undesirable sleep behavior by a stimulus universally sensed by most patients where the stimulation device avoids significantly changing sleep states and certainly avoids waking a patient.

Another advantage of the present invention is the ability to be precisely controllable so as to provide precise doses of stimulation to a sleep disorder patient.

Another advantage of the present invention is that it is comfortable to wear so as to further its ability to avoid alteration of sleep states.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventor also contemplates examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A sleep therapy transducer for a patient, the sleep therapy transducer comprising:
a pyro/piezoelectric film configured to detect respiration information of the patient and configured to provide a stimulation to the patient;
a first electrode attached to a first major surface of the pyro/piezoelectric film;
a second electrode attached to a second major surface of the pyro/piezoelectric film;
wherein the first and second electrodes are configured to transmit the detected respiration information from the pyro/piezoelectric film to a closed loop neuromodulator and to receive stimulation energy from the closed loop neuromodulator; and
wherein the stimulation is provided to the patient using the received stimulation energy.

2. The sleep therapy transducer of claim 1, including a first adhesive to attach the first electrode to the first major surface of the pyro/piezoelectric film.

3. The sleep therapy transducer of claim 1, including a second adhesive to attach the second electrode to the second major surface of the pyro/piezoelectric film.

4. The sleep therapy transducer of claim 3, wherein the first and second adhesives include a conductive adhesive.

5. The sleep therapy transducer of claim 1, including a first wire coupled to the first electrode and a second wire coupled to the second electrode, the first and second wires configured to transmit detected respiration information from the pyro/piezoelectric film to a closed loop neuromodulator and to transmit stimulation energy from the closed loop neuromodulator to the pyro/piezoelectric film.

6. The sleep therapy transducer of claim 5, including:
a first adhesive film substantially covering the first major surface of the pyro/piezoelectric film, the first adhesive film to secure the first wire to the first major surface of the pyro/piezoelectric film, and
a second adhesive film substantially covering the second major surface of the pyro/piezoelectric film, the second adhesive film to secure the second wire to the second major surface of the pyro/piezoelectric film.

7. The sleep therapy transducer of claim 6, wherein the first adhesive film is a double sided adhesive film configured to secure the sleep therapy transducer to the patient.

8. The sleep therapy transducer of claim 7, wherein the double sided adhesive film is configured to provide acoustic impedance matching between the sleep therapy transducer and the patient's skin.

9. The sleep therapy transducer of claim 1, wherein the pyro/piezoelectric film includes a metalized polyvinylidene fluoride (PVDF) film.

10. The sleep therapy transducer of claim 1, wherein the sleep therapy transducer is configured to attach behind an ear of the patient.

11. The sleep therapy transducer of claim 1, wherein the sleep therapy transducer is configured to attach near a lip of the patient.

12. The sleep therapy transducer of claim 1, wherein the sleep therapy transducer is configured to attach over the patient's nose.

13. The sleep therapy transducer of claim 1, wherein the sleep therapy transducer is configured to attach to the patient's neck.

14. The sleep therapy transducer of claim 1, wherein the sleep therapy transducer is configured to emit at least one of a tactile stimulation or an acoustic stimulation.

15. A sleep therapy transducer for a patient, the sleep therapy transducer comprising:
a pyro/piezoelectric film configured to detect respiration information of the patient and configured to provide a stimulation to the patient;
a first electrode attached to a first major surface of the pyro/piezoelectric film;
a second electrode attached to a second major surface of the pyro/piezoelectric film;
a first conductive adhesive to attach the first electrode to the first major surface of the pyro/piezoelectric film;
a second conductive adhesive to attach the second electrode to the second major surface of the pyro/piezoelectric film;
a first wire coupled to the first electrode and a second wire coupled to the second electrode;
a first adhesive film substantially covering the first major surface of the pyro/piezoelectric film, the first adhesive film to secure the first wire to the first major surface of the pyro/piezoelectric film;
a second adhesive film substantially covering the second major surface of the pyro/piezoelectric film, the second adhesive film to secure the second wire to the second major surface of the pyro/piezoelectric film;
wherein the first and second wires are configured to transmit detected respiration information from the pyro/piezoelectric film to a closed loop neuromodulator, and to transmit stimulation energy from the closed loop neuromodulator to the pyro/piezoelectric film; and
wherein the first adhesive film is a double sided adhesive film configured to secure the sleep therapy transducer to the patient.

16. A method comprising:
detecting respiration information of a patient using a sleep therapy transducer attached to the patient, the sleep therapy transducer including a pyro/piezoelectric film;
transmitting the detected respiratory information from the sleep therapy transducer to a closed loop neuromodulator using a first electrode attached to a first major surface of the pyro/piezoelectric film and a second electrode attached to a second major surface of the pyro/piezoelectric film;
receiving stimulation energy from the closed loop neuromodulator using the first and second electrodes; and
providing a stimulation to the patient using the received stimulation energy.

17. The method of claim 16, wherein the transmitting the respiration information includes transmitting the respiration information using a using a single wire pair coupled to the first and second electrode.

18. The method of claim 17, wherein the receiving the stimulation energy includes receiving the stimulation energy using the single wire pair coupled to the first and second electrode.

19. The method of claim 16, wherein the providing the stimulation includes providing the stimulation behind an ear of the patient.

20. The method of claim 16, wherein the providing the stimulation includes providing the stimulation near a lip of the patient.

21. The method of claim 16, wherein the providing the stimulation includes providing the stimulation to the patient's nose.

22. The method of claim 16, wherein the providing the stimulation includes providing the stimulation to the patient's neck.

23. The method of claim 16, wherein the providing the stimulation includes providing at least one of tactile stimulation or acoustic stimulation.
